(19) United States
(12) Plant Patent Application Publication
(10) Pub. No.: US 2018/0160584 P1
(43) Pub. Date: Jun. 7, 2018

(54) Avocado variety named 'KB1'

(50) ;

(71) Applicant: Klaus Bederski Lehmann, Lima (PE)

(72) Inventor: Klaus Bederski Lehmann, Lima (PE)

(21) Appl. No.: 15/530,161

(22) Filed: Dec. 6, 2016

(51) Int. Cl. A01H 5/08

(52) U.S. Cl. PLT/200

(57) ABSTRACT

A new avocado variety, ‘KB1 ’, was discovered in Chincha, Peru. The ‘KB1 ’ variety when used as a rootstock is notable for its prolific and vigorous feeder root growth ability that results in rapid canopy volume and early productivity of the ‘Hass’ avocado scion cultivar, and induces larger fruit sizes of the ‘Hass’ cultivar and is an alternative for locations where ecologic conditions tend to produce small fruit.

Latin name of the genus and species: Persea americana Mill.

Varietal denomination: ‘KB1’.

### BACKGROUND OF THE INVENTION

Historically, avocado scion varieties were propagated by grafting them over seedling rootstocks of three origins: Mexican, Guatemalan and West Indian. Each growing area used the alternative best suited to its proper ecologic conditions. California chose seedling rootstocks of Mexican origin, typically ‘Topa Topa’ (unpatented). Florida chose seedling rootstocks of West Indian origin, typically ‘Waldin’ (unpatented). Seedling rootstocks proved to be variable in their morphological and productive characteristics. The avocado industry demanded more uniformity in growth and productivity of orchard trees than trees on seedling rootstocks were able to provide.

The search for an answer led to the development of clonal rootstocks where all trees in an orchard were genetically identical to each other. Eventually, ‘Duke 7’ (unpatented) became the leading clonal avocado rootstock for California. This clonal rootstock, and more recently also the ‘Merensky 2’ (U.S. Plant Pat No. 15,309) clonal rootstock, have been introduced for cultivation worldwide. Clonal avocado rootstocks, like seedling avocado rootstocks, are not necessarily best suited to the particular soil-water-climate conditions of all avocado growing regions world-wide.

Currently, the main avocado variety grown in the word is ‘Hass’ (U.S. Plant Pat. No. 139, the contents of which are incorporated herein by reference). The main markets have year-round supply of ‘Hass’ avocados and end consumers are very used to buying ‘Hass’. Many other avocado varieties have been created and patented in the last twenty years, but none of them has been able to obtain important interest from growers. The main reason for the lack of interest is that the market still prefers ‘Hass’.

### SUMMARY OF THE INVENTION

‘KB1’ is a new and distinct variety of avocado tree Persea americana Mill. ‘KB1’ is a clonal avocado rootstock that has rapid uniform growth and early productivity.

In the mid-1990s during a weekend family tour into a remote rural area located in Andean Highlands east of the city of Lima, Peru Mr. Bederski Lehmann saw a large well-foliated ‘Fuerte’ (unpatented) cultivar avocado tree growing in a roadside orchard where most of the surrounding trees were unproductive due to poor canopy growth.

Closer observation showed that poor canopy growth in this orchard was due to the interaction of three factors: incorrect irrigation, mechanical root damage caused by plowing or discing, and pathology caused by the endemic presence of the root rot fungus Phytophthora cinnamomi Rands. This one tree had the remarkable ability not to be damaged by any one of the abovementioned factors.

The rootstock of this one outstanding tree had watersprouts which the inventor cut in order to propagate this outstanding source of avocado genetics and grafted them as scions over Mexican rootstocks that were growing at his nursery on his property. The intention was to observe and eventually use this new genetic material as a new rootstock alternative source for avocado propagation. As years passed, these grafted trees became productive. Their fruits had normal viable seeds which were planted to be used as seedling rootstocks for avocados.

The seedlings that grew from these seeds did not show the genetic uniformity that is required by nurseries and by growers of the avocado industry. Their extreme variability in vigor as well as in the color and leaf characteristics suggested that this rootstock source had an unexpected genetic complexity. At this point the inventor decided to use it as a source of genetic material for clonal instead of for seed propagation. Clonal propagation guarantees genetic uniformity.

The Foundation Tree of the ‘KB1’ clonal avocado rootstock is located on the property of the inventor, Mr. Bederski Lehmann in Chincha, Peru. This one tree has provided the material for ‘Hass’ avocados on ‘KB1’ clonal that has thus far been grown.

‘Hass’ avocado on ‘KB1’ clonal rootstock was planted for limited trial at the inventor's property in 2010 and in 2013. ‘Hass’ on ‘KB1’ clonal rootstock at this location soon proved to be a superior alternative exhibiting uniform rapid growth and early productivity when compared to conventional Hass on seedling rootstocks.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying photographs show specimens of the tree and plant parts of the new ‘KB1’ variety.

FIG. 1 is a photograph of the ‘KB1’ foundation tree growing in Chincha, Peru;

FIG. 2 is a photograph depicting lenticels of the ‘KB1’ variety;

FIG. 3 is a photograph depicting flush foliage of the ‘KB1’ variety;

FIG. 4 is a photograph depicting inflorescence of the ‘KB1’ variety;

FIG. 5 is a photograph depicting an external view of the fruit of the ‘KB1’ variety;

FIG. 6 is a photograph depicting an internal view of the fruit of the ‘KB1’ variety, with and without the seed;

FIG. 7 is a graph depicting the comparison of tree canopy diameter for the ‘KB1’ variety as compared to other avocado varieties;

FIG. 8 is a graph depicting the yield of fruit for the ‘KB1’ variety as compared to other varieties; and

FIG. 9 is a graph depicting the percentage of defective fruit of the ‘KB1’ variety as compared to other avocado varieties.

### DETAILED BOTANICAL DESCRIPTION OF THE VARIETY

The following detailed botanical description is based on observations of a ‘KB1’ rootstock variety which was taken from an approximately sixteen-year-old mature tree, planted in 2000 in Chincha, Peru, and evaluations conducted in 2016 for ‘Hass’ on ‘KB1’ clonal rootstock planted between 2010 and 2014 in Chincha, Peru.

In those instances where precise color assessment could be made, references are to The Royal Horticultural Society (R.H.S.) Colour Chart Sixth Edition (2015). In other instances, generally, color terms are used in accordance with an ordinary dictionary significance. Other descriptions are taken from the generally recognized description chart found in the Graphic Handbook for the Description of Avocado Varieties published by SAGARPA in January, 2010 (ISBN: 978-607-12-0106-5) (“Graphic Handbook”).

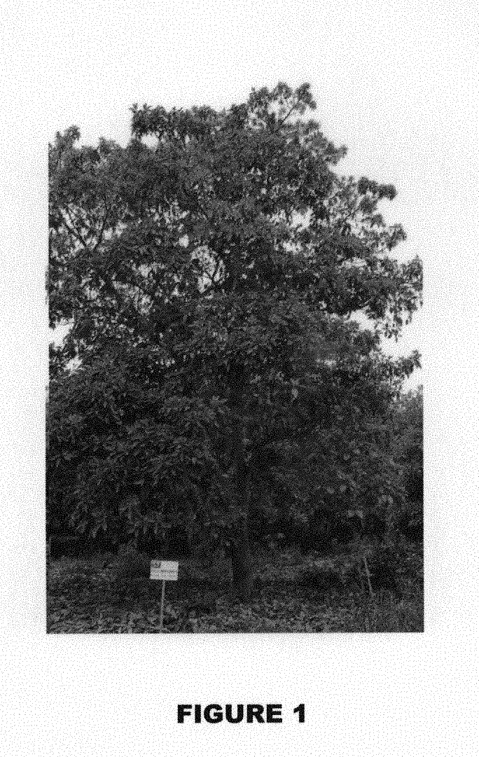

With reference to FIG. 1, the ‘KB1’ foundation tree is shown which was planted in 2000 in Chincha, Peru. The tree is now approximately eight meters tall and six meters in diameter. An aspect of ‘KB1’ is that it has rapid uniform growth. The ‘KB1’ has a generally upright tree shape. The color of a one-year-old branch is strong yellow green (RHS 144B). Young shoots have a soft, juicy texture. The color of expanding apical leaves of young shoots is moderate yellowish brown (RHS N199C).

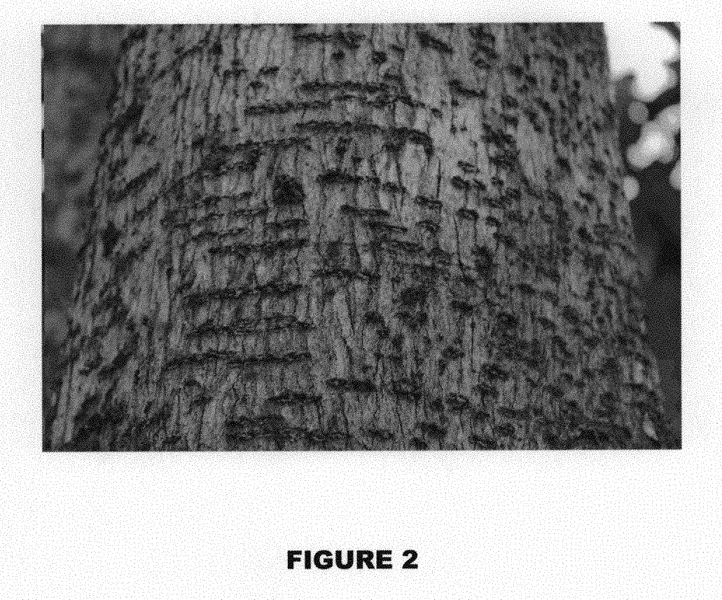

As illustrated in FIG. 2, the lenticels found on the ‘KB1’ tree are big, long and horizontal. The color of the lenticels (one-year-old bark) are moderate brown (RHS 200C). The bark texture of a one-year-old branch is smooth. One-year-old bark has a moderate orange yellow color (RHS 165C), while the oldest bark growth has a dark grayish yellow color (RHS 199D).

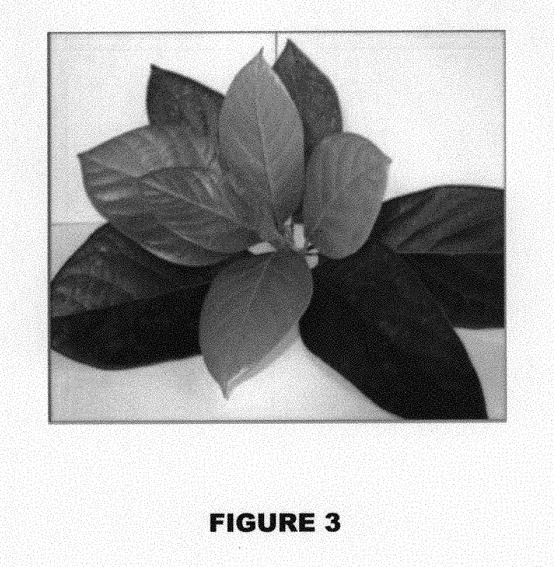

FIG. 3 illustrates typical flush foliage of ‘KB1’. The young shoot color is moderate yellowish brown (RHS N199C). The leaf blade anise aroma is strong, indicating Mexican origin. The Graphic Handbook uses the presence or absence of anise smell in the leaf blade as an indication of the tree origin; West Indian having no smell, whereas Mexican has anise smell.

The leaf arrangement is upwards relative to shoot (Graphic Handbook). Mature leaves have a weak undulation margin. The leaf venation pattern of a mature, fully expanded leaf is asymmetrical, as illustrated. An upper surface vein has a brilliant greenish yellow color (RHS 151D). The veins of a mature, fully-expanded leaf are level (Graphic Handbook). A mature, fully expanded, leaf typically has an intermediate number of secondary veins between 14.5 and 17.5 secondary veins on the leaf blade.

The density of pubescence on the lower surface of a mature leaf is medium. Medium density of pubescence on the lower surface of a mature leaf is shown in the Graphic Handbook under element 17. Texture of upper surface of mature leaves is medium glossy.

Twisting along the whole length of the leaf blade of a mature leaf is absent. The shape of the apex of the leaf blade is acuminate. The leaf base shape of a mature, fully expanded leaf is equilateral. The leaf blade length is medium (16.1 cm - 18.4 cm) (Graphic Handbook). The leaf blade width of a mature, fully expanded leaf is also medium (6.6 cm - 7.6 cm) (Graphic Handbook).

The color of the upper surface of a mature fully expanded leaf is greyish olive green (RHS NN 137B). The color of the lower surface of a mature, fully expanded leaf is pale green (RHS N 138C). The color of tender, fully grown leaves is moderate olive green (RHS 146A).

The length of a mature, fully expanded leaf petiole is medium (between 3.9 cm and 4.62 cm) (Graphic Handbook). The diameter of a mature, fully expanded leaf petiole is approximately 2.5 mm. The color of a mature, fully expanded leaf petiole is moderate yellow green (RHS 146C).

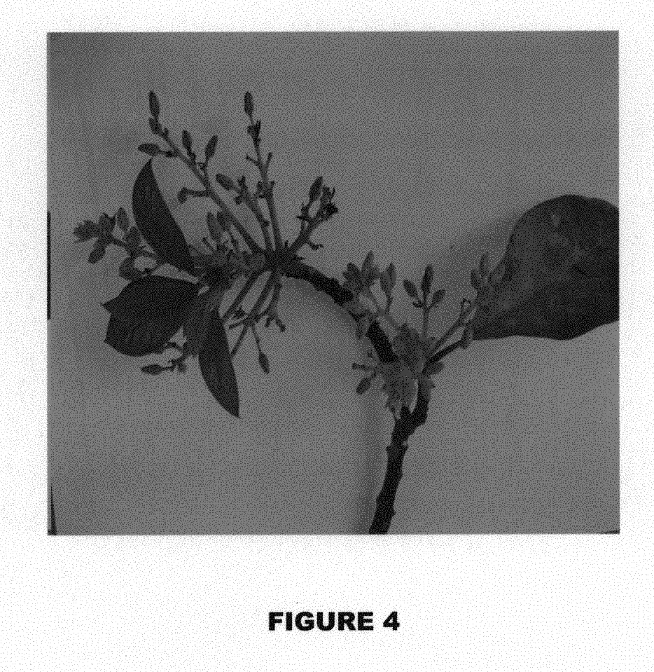

With reference now to FIG. 4, typical inflorescence of the ‘KB1’ variety is shown. The cutting illustrated measures approximately 4.09 cm-6.53 cm. The ‘KB1’ variety has a B flowering type. The flower fragrance of a fully opened flower is absent. The perianth (sepals and petals) of a newly, fully opened flower is borne in two whorls of three perianth lobes. The length of a fully developed flower bud is approximately nine millimeters, and the diameter is approximately five millimeters. There are nine stamen of a fully opened flower. The pistil of the fully opened flower has one each of a stigma, style and ovary. The ovary is superior. The color of a fully developed flower bud is brilliant yellow green (RHS 150C). The petal color of the fully opened flower, on both surfaces, is light yellow green (RHS 154C).

In its native Peru, the spring bloom period extends from early August through mid-October. ‘KB1’ rarely has off-season flowers. Fruit set is poor and only a very few fruits ripen.

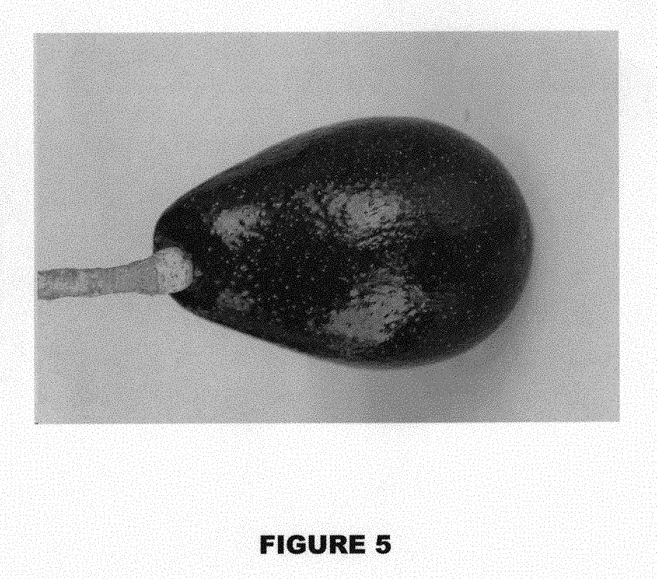

With reference to FIG. 5, an exterior view of a typical fruit of the ‘KB1’ variety is shown. The diameter of the fruit measures 6.60 cm-8.33 cm on average so as to have a relatively small mature fruit diameter, such as an ‘Edranol’. The length of the fruit on average measures 10.71 cm-13.23 cm, which is short, as in ‘Edranol’. However, the mature fruit ratio length/diameter is medium, as in ‘Topa Topa’. The average fruit weight, taken from an average of six mature fruit, is 159 grams.

The color of the fruit skin as it starts to change color is strong yellow green (RHS 143A). Phase 2 in color change of the fruit skin, the color is dark red (RHS 183B). The mature fruit skin color is dark red (RHS 187A). The fruit skin color of a fully ripe fruit is black (RHS 203B).

With continuing reference to FIG. 5, the length of the fully expanded pedicel is short, between 0.99 and 1.45 cm. The shape of the pedicel is conical and does not exhibit a “nailhead” at the junction. The thickness of the fully expanded pedicel compared to peduncle at junction is thicker, approximately 2 mm. The pedicel surface texture is wrinkled. The fully expanded peduncle is approximately 3.56 cm, with a diameter of approximately 3.2 mm. The color of the fully expanded peduncle is strong greenish-yellow (RHS 151A).

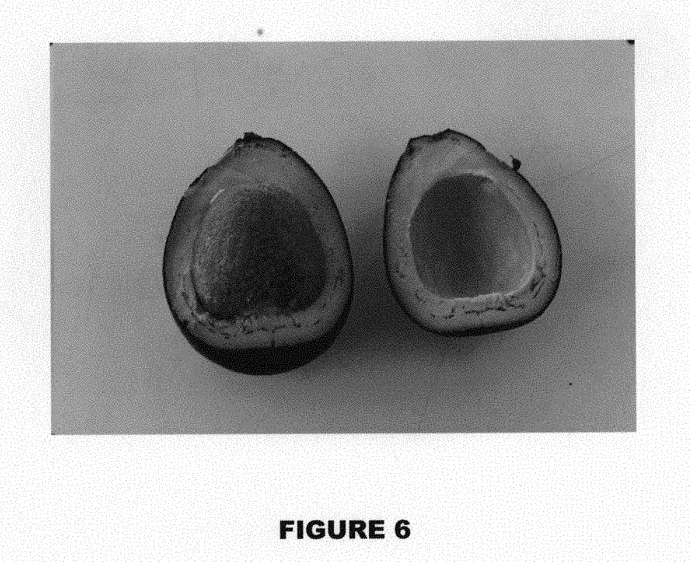

FIG. 6 is a typical internal view of the ‘KB1’ variety fruit, illustrating with and without the seed. The shape of the seed in longitudinal section is generally ovate. The seed diameter at the widest equatorial point (taken from an average of four seeds) is 3.175 cm. The average seed length at polar ends, taken from the four seeds, is 4.445 cm. The average weight of the seed is 29.25 grams. The seed color is pale orange yellow (RHS 24D).

With continuing reference to FIG. 6, the color of the flesh of a fully ripe fruit is light yellow-green (RHS 145 B). The lenticel color of the fully ripe fruit is dark red (RHS 187B). As illustrated in FIGS. 5 and 6, the color of the ripe fruit is dark greyish purple (RHS 202A) or black (RHS 203B). The thickness of the skin of the ripe fruit is very thin.

An experimental block of ‘Hass’ avocado was planted in March 2014 at the inventor's property using four (4) rootstock sources: ‘KB1’ clonal, West Indian ‘Waldin’ seedlings, seedling selection and (hybrid) Mexican×Guatemalan ‘Zutano’ (unpatented) seedlings. Data taken in 2016 has shown that statistical differences do exist in canopy volume, early productivity, fruit weight (caliper) and cosmetic external quality. All of these measurements show ‘KB1’ clonal as the superior rootstock choice under the soil-water-climate conditions of this location. The following is the data of the ‘KB1’ avocado rootstock compared to other standard avocado rootstocks, as mentioned above, during the evaluation process in Peru and California.

TABLE 1

| Rootstock | Canopy Diameter (Meters) |
|---|---|
| ‘Waldin’ | 5.86 |
| Clonal ‘KB1’ | 7.68 |
| ‘Zutano’ | 5.96 |
| Seedling Selection | 5.55 |

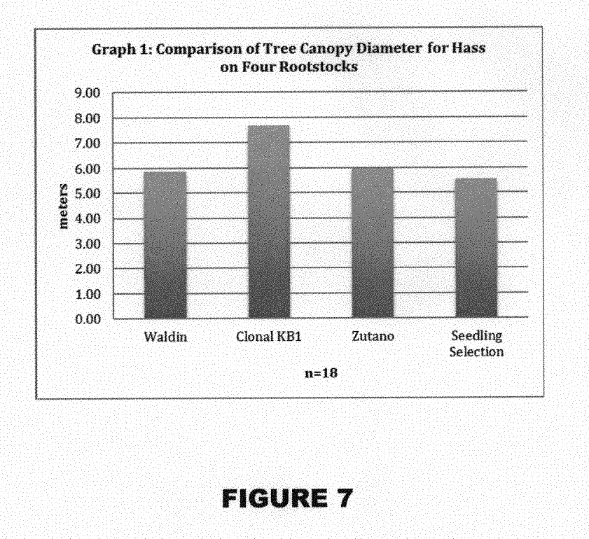

Table 1 above is the average diameter of the tree canopy for ‘Hass’ on the four different rootstocks, including the ‘KB1’ variety. FIG. 7 is a corresponding graph illustrating a comparison of the tree canopy diameter for ‘Hass’ for the four rootstocks of Table 1.

TABLE 2

| | No. of | Total Yield | Average Yield/ |
|---|---|---|---|
| Rootstock | Trees | (KG) | Tree (KG) |
| Clonal ‘KB1’ | 92 | 728.64 | 7.92 |
| ‘Waldin’ | 80 | 551.43 | 6.89 |
| ‘Zutano’ | 84 | 522.82 | 6.22 |
| Seedling Selection | 73 | 58.64 | 0.80 |

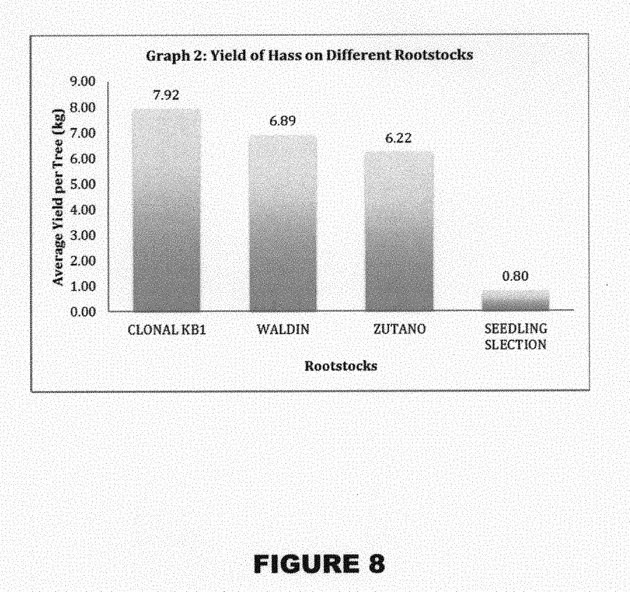

Table 2 is a table illustrating the yield of ‘Hass’ on the different rootstocks. FIG. 8 is a graph depicting the yield of ‘Hass’ on the different rootstocks.

TABLE 3

| Rootstock | Fruit No. | Defective Fruit No. | Defective Fruit % |
|---|---|---|---|
| Clonal ‘KB1’ | 1483 | 16 | 1.08% |
| ‘Zutano’ | 955 | 87 | 9.11% |
| Seedling Selection | 218 | 28 | 12.84% |
| ‘Waldin’ | 1254 | 171 | 13.64% |

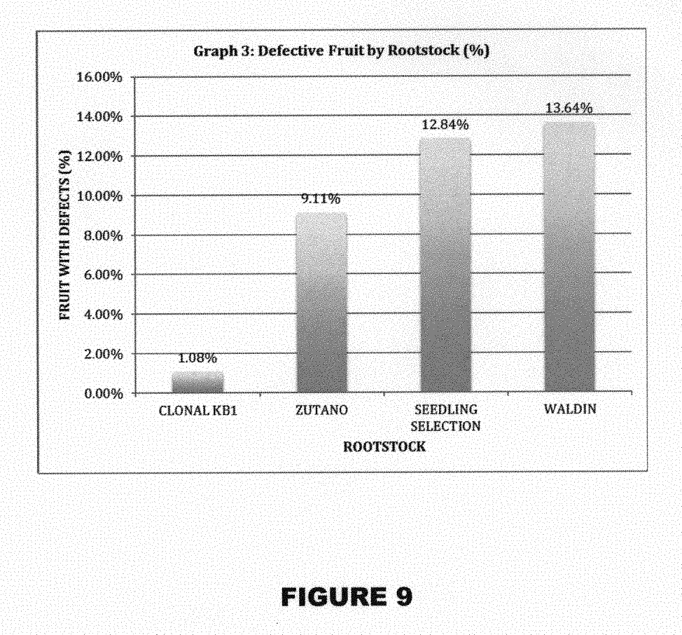

Table 3 illustrates the incidents of defective fruit for the ‘KB1’ variety, as compared to the other three different rootstocks, and FIG. 9 is a graph depicting the percentage of defective fruit by rootstock.

As illustrated in Table 4 below, one hundred fruit per rootstock, including the ‘KB1’ variety as compared to the other varieties, from two-year-old trees were randomly selected and evaluated in order to classify the fruits by fruit counts (CODEX NORM).

TABLE 4

| | Count (g) | | | | | | | | | |
|---|---|---|---|---|---|---|---|---|---|---|
| | 8 | 10 | 12 | 14 | 16 | 18 | 20 | 22 | 24 | 26 |
| Rootstock | (460-575) | (365-460) | (305-365) | (265-305) | (235-265) | (210-235) | (190-210) | (170-190) | (155-170) | (145-155) |
| Waldin | | 2% | 12% | 23% | 19% | 32% | | 12% | | |
| Clonal KB1 | | 3% | 39% | | 38% | | 16% | | | 4% |
| Zutano | 1% | 2% | | 24% | 33% | | 30% | | | |
| Seedling Selection | 1% | 2% | 13% | | 44% | 32% | | | 8% | |

As shown in Table 5 below, the yield of the ‘Hass’ cultivar on the four rootstocks, including the ‘KB1’ variety, was measured in the two-year-old trees, and the yield extrapolated to one hectare.

TABLE 5

| | Yield per | Distance | Total number | Yield per |
|---|---|---|---|---|
| Rootstock | tree (kg) | (m) | of trees per ha | ha (ton) |
| Clonal KB1 | 7.76 | 6 × 4 | 416 | 3.23 |
| Waldin | 6.89 | 6 × 4 | 416 | 2.87 |
| Zutano | 6.2 | 6 × 4 | 416 | 2.58 |
| Seedling Selection | 0.79 | 4 × 3 | 833 | 0.66 |

‘KB1’ is to be used commercially as a rootstock. As can be seen from the above, the new and distinct avocado variety ‘KB1’ when used as a rootstock confers to the tree a high yield and high vigor when topworked to ‘Hass’. Moreover, the ‘KB1’ avocado rootstock cultivar variety is notable for its prolific and vigorous feeder root growth ability that results in rapid canopy volume and early productivity of the ‘Hass’ avocado scion cultivar. The ‘KB1’ variety induces larger fruit sizes of the ‘Hass’ cultivar and is an alternative for locations where ecologic conditions can produce small fruit, such as in well-drained alkaline soils in low rainfall conditions.

1. A new and distinct avocado rootstock, as herein illustrated and described.
